(19) United States
(12) Patent Application Publication
(10) Pub. No.: US 2026/0276770 A1
(43) Pub. Date: Sep. 17, 2026

(54) ACCELERATION OF MAGNETIC RESONANCE ELASTOGRAPHY (MRE) USING DISTRIBUTED ENCODING

(71) Applicant: University of Delaware, Newark, DE (US)

(72) Inventors: Curtis Laurence Johnson, Newark, DE (US); Alexander Cerjanic, Newark, DE (US); Mary Kramer, Newark, DE (US)

(73) Assignee: University of Delaware, Newark, DE (US)

(21) Appl. No.: 19/582,309

(22) PCT Filed: Oct. 25, 2024
(86) PCT No.: PCT/US2024/052930

§ 371 Date: Apr. 27, 2026

Related U.S. Application Data
(60) Provisional application No. 63/593,743, filed on Oct. 27, 2023.

(51) Int. Cl. G01R 33/563; A61B 5/00; A61B 5/055; G01R 33/565

(52) U.S. Cl. CPC G01R 33/56358; A61B 5/0042; A61B 5/055; G01R 33/56509

(57) ABSTRACT

A method for generating a map indicative of mechanical properties of subject tissue using an MRI system includes performing an MRE scan on the subject tissue with the MRI system and obtaining and processing MRE data related to the subject tissue via a control device. The control device is configured to perform a plurality of operations including: encoding a series of motions and obtaining MRE data samples in a plurality of directions at a predetermined number of time points, constructing a plurality of phase images based upon the obtained MRE data samples, and having a number of phase images in a range from 7 to 24, estimating shear displacement fields in the subject tissue based on the plurality of phase images, wherein estimation includes an optimization performed according to an EDGE matrix, and generating the map indicative of the subject tissue's mechanical properties via an inversion algorithm.

### CROSS-REFERENCE TO RELATED APPLICATIONS

This application claims priority to U.S. Provisional Application No. 63/593,743, filed Oct. 27, 2023, the content of which is incorporated herein by reference in its entirety for all purposes.

### REFERENCE TO U.S. GOVERNMENT SUPPORT

This invention was made with government support under Grant Nos. R01AG058853A and R01EB027577A, awarded by the National Institutes of Health, with other funding information U01NS112120, awarded by the National Institutes of Health. The United States has certain rights in the invention.

### FIELD OF THE INVENTION

Aspects of the invention relate generally to magnetic resonance elastography (MRE) and magnetic resonance imaging (MRI) systems. More particularly, aspects of the invention relate to method and systems for generating a map indicative of mechanical properties of a subject tissue using an MRI system.

### BACKGROUND OF THE INVENTION

The mechanical properties of tissues (e.g. human brain tissue) can be indicative of overall health, including any underlying medical conditions, and as such have been used in many diagnostic capacities. Magnetic resonance elastography (MRE) is a non-invasive, in vivo, magnetic resonance imaging (MRI) technique which provides maps of mechanical properties (e.g., stiffness) by imaging a displacement of subject tissue in response to mechanical actuation. Namely, MRE uses mechanical actuation to cause micron-level displacements of tissue, which are imaged using motion encoding gradients. These MRE-derived mechanical properties can provide insight into microstructural changes in the tissue that may be indicative of conditions related to aging and disease progression.

Conventional MRE typically encodes motion in each of three cardinal axes, i.e. x, y, z, and with repeated scans to capture waves in time, thereby yielding complex harmonic displacements throughout the entire tissue after application of a temporal Fourier transform. Each direction and time point pair is a separate image acquired during the scan so the length of the entire MRE scan is dependent on the number of image encodings (directions×time points) acquired. Typical techniques require sampling separately in three orthogonal directions with 4 to 8 phase offsets and two gradient polarities, resulting in a total of 24 to 48 images, which exceeds the theoretical minimum samples needed to generate an image (e.g. minimum is 7: real and imaginary components of 3 directions of motion—x, y, z—and background phase). Accordingly, some constraints of conventional MRE and MRI techniques include relatively long durations of scan times which are necessary to encode three directions of motion across time, thereby limiting clinical adoption of said techniques and making said techniques susceptible to movement by the subject during imaging.

Therefore, there remains a desire for improvements in systems and methods for imaging of subject tissue in response to mechanical actuation, particularly for generating a map indicative of mechanical properties of the subject tissue using an MRI system.

### SUMMARY OF THE INVENTION

According to an aspect of the invention, a method for generating a map indicative of mechanical properties of a subject tissue using a magnetic resonance imaging (MRI) system includes performing a magnetic resonance elastography (MRE) scan on the subject tissue with the MRI system. The method also includes obtaining and processing MRE data related to the subject tissue via a control device coupled to the MRI system. The control device has a controller configured to perform a plurality of operations in accordance with machine readable instructions stored in a digital memory. The plurality of operations comprises encoding a series of motions and obtaining MRE data samples in a plurality of directions at a predetermined number of time points. The encoding motion and obtaining samples are performed according to an Elastography with Distributed, Generalized Encoding (EDGE) matrix. The plurality of operations also includes constructing a plurality of phase images based upon the obtained MRE data samples, and the plurality of phase images comprises a number of phase images in a range from 7 to 24. The plurality of operations further comprises estimating shear displacement fields in the subject tissue based on the plurality of phase images, wherein estimation comprises an optimization performed according to an Elastography with Distributed, Generalized Encoding (EDGE) matrix. The plurality of operations further includes generating the map indicative of the subject tissue's mechanical properties via an inversion algorithm.

According to another aspect of the invention, a magnetic resonance imaging (MRI) system for generating a map indicative of a subject tissue's mechanical properties includes a magnet system configured to generate a polarizing magnetic field to the subject tissue. The system also includes a plurality of gradient coils configured to apply a magnetic field gradient to the polarizing magnetic field; a radio frequency (RF) system configured to apply an excitation field to the subject tissue; and a driver configured to deliver a stress to the subject for propagating mechanical waves through the subject tissue. The system also comprises a control device coupled to the MRI system. The control device has a controller configured to perform a plurality of operations in accordance with machine readable instructions stored in a digital memory. The plurality of operations comprises encoding a series of motions and obtaining MRE data samples in a plurality of directions at a predetermined number of time points. The encoding motion and obtaining samples are performed according to an Elastography with Distributed, Generalized Encoding (EDGE) matrix. The plurality of operations also includes constructing a plurality of phase images based upon the obtained MRE data samples, and the plurality of phase images comprises a number of phase images in a range from 7 to 24. The plurality of operations further comprises estimating shear displacement fields in the subject tissue based on the plurality of phase images, wherein estimation comprises an optimization performed according to an Elastography with Distributed, Generalized Encoding (EDGE) matrix. The plurality of operations also includes generating the map indicative of the subject tissue's mechanical properties via an inversion algorithm.

### BRIEF DESCRIPTION OF THE DRAWINGS

The foregoing summary and the following description will be better appreciated and understood in conjunction with the non-limiting examples illustrated in the attached drawing figures, of which:

FIG. 1 depicts a schematic diagram of an MRI system in accordance with aspects of the invention;

FIG. 2 depicts an exemplary method for generating a map indicative of mechanical properties of a subject tissue using an MRI system in accordance with aspects of the invention;

FIG. 3A depicts a generalized encoding matrix in accordance with prior art;

FIG. 3B depicts an enlarged portion of FIG. 3A;

FIG. 4A depicts an exemplary embodiment of a generalized encoding matrix in accordance with aspects of the invention;

FIG. 4B depicts an enlarged portion of FIG. 4A;

FIG. 4C depicts a graph showing encoding samples along an optimized set of gradient vector directions in accordance with aspects of the invention;

FIG. 4D depicts an encoding matrix as described in the graph of FIG. 4C, showing each sample as a row of the matrix, which contains information about each direction (column of the matrix) described by a line in the graph of FIG. 4C, in accordance with aspects of the invention;

FIGS. 5-6 depict results from test performed on simulated brain data in accordance with validation testing of the system of FIG. 1 and method of FIG. 2;

FIG. 7 depicts results from tests performed on phantom data in accordance with validation testing of the system of FIG. 1 and method of FIG. 2;

FIG. 8 depicts results from tests performed on three human subjects' in accordance with validation testing of the system of FIG. 1 and method of FIG. 2; and

FIGS. 9A-9B depict results from tests performed on data with image volumes removed after acquisition in accordance with validation testing of the system of FIG. 1 and method of FIG. 2.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention. Additionally, various forms and embodiments of the invention are illustrated in the figures. It will be appreciated that the combination and arrangement of some or all features of any of the embodiments with other embodiments is specifically contemplated herein. Accordingly, this detailed disclosure expressly includes the specific embodiments illustrated herein, combinations and sub-combinations of features of the illustrated embodiments, and variations of the illustrated embodiments.

In the following detailed description, numerous specific details are set forth by way of examples in order to provide a thorough understanding of the relevant design features. However, it should be apparent to those skilled in the art that the present design features may be practiced without such details. In other instances, well known methods, procedures, components, and circuitry have been described at a relatively high-level, without detail, in order to avoid unnecessarily obscuring aspects of the present design.

In general, aspects of the invention relate to systems and methods for generating a map indicative of mechanical properties of a subject tissue using a magnetic resonance imaging (MRI) system. The systems and methods described can include an ability to provide robust displacement estimation with reduced sampling, achieve noise reduction through regularization schemes tailored to the MRE signal, and estimate rigid body displacement by handling phase wrapping. Advantages of embodiments of the claimed invention includes permitting a reduced sampling requirement (e.g. undersampling), thereby (1) reducing the overall time needed to acquire MRE data (e.g. MRE data that is useful for clinical adoption and applications on children) and (2) in cases where images associated with specific samples are corrupted from excessive motion of the subject, such as might occur with children, displacements can still be estimated if said corrupted images are rejected and/or deleted.

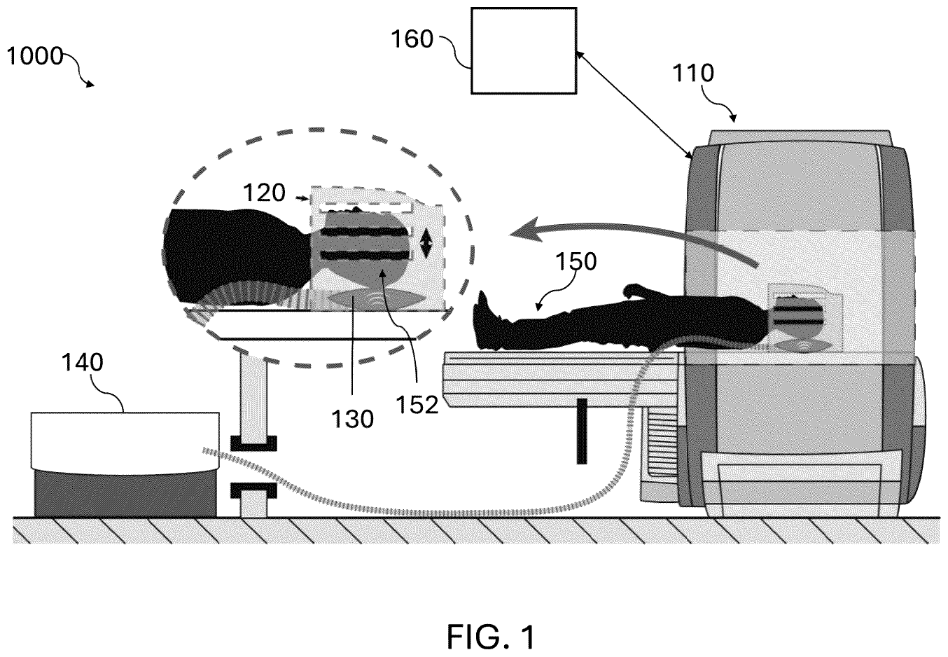

Referring to FIG. 1, a schematic representation is provided of an exemplary magnetic resonance imaging (MRI) system for generating a map indicative of a subject tissue's mechanical properties. In general, an exemplary embodiment of the MRI system, such as MRI system 1000, includes a magnet system 110, a plurality of gradient coils 120, a radio frequency (RF) system 130, a driver 140, and a control device 160.

The magnet system 110 is configured to generate a polarizing magnetic field to the subject tissue 152 of patient 150. The plurality of plurality of gradient coils 120 are configured to apply a magnetic field gradient to the polarizing magnetic field generated by the magnet system 110. The RF system 130 is configured to apply an excitation field to the subject tissue 152 (e.g. brain of patient 150). The driver 140 is configured to deliver a stress to the subject 150 for propagating mechanical waves through the subject tissue 152.

The control device 160 is coupled to the MRI system 1000. In an exemplary embodiment, the control device 160 has a controller configured to perform a plurality of operations in accordance with machine readable instructions stored in a digital memory. The plurality of operations includes encoding a series of motions and obtaining MRE data samples in a plurality of directions at a predetermined number of time points. In exemplary aspects of the invention, the encoding motion and obtaining samples is performed according to an Elastography with Distributed, Generalized Encoding (EDGE) matrix. The EDGE matrix includes encoding and obtaining samples along an optimized set of gradient vector directions distributed in a 3-dimensional gradient vector space across a surface of a sphere. Additionally, the plurality of operations includes constructing a plurality of phase images based upon the obtained MRE data samples. In an exemplary embodiment, the plurality of phase images includes a number of phase images in a range from 7 to 24. Further, the plurality of operations includes estimating shear displacement fields in the subject tissue 152 based on the constructed plurality of phase images. Estimation of the shear displacement fields includes performing an optimization according to the EDGE matrix. Still further, the plurality of operations includes generating the map indicative of the subject tissue's 152 mechanical properties via an inversion algorithm.

Additional details of the plurality of operations of system 1000 will now be discussed below in the context of method 2000.

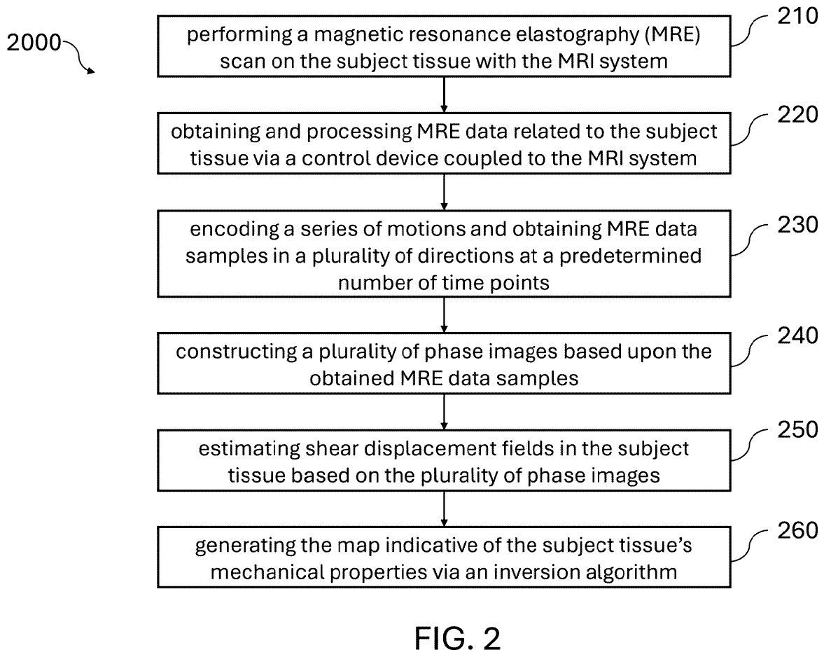

Turning now to FIGS. 1 and 2, an exemplary method 2000 of generating a map indicative of mechanical properties of a subject tissue using an MRI system, such as the MRI system 1000, is disclosed. Generally, method 2000 includes step 210 of performing a magnetic resonance elastography (MRE) scan on the subject tissue 152 with the MRI system 1000 and step 220 of obtaining and processing MRE data related to the subject tissue 152, such as via a control device 160 associated with or coupled to one or more components of the MRI system 1000. In an exemplary embodiment, the control device 160 has a controller configured to perform a plurality of operations in accordance with machine readable instructions stored in a digital memory.

In an exemplary embodiment of step 210, performing the MRE scan includes the one or more of the following sub-steps of: applying a polarizing magnetic field to the subject tissue 152; irradiating the subject tissue 152 with a radio frequency (RF) excitation field for producing transverse magnetization in spins in the subject tissue 152; applying a magnetic field gradient to the spins which alternates in polarity; and applying a stress to the subject tissue 152 in cooperation with alternation of the magnetic field gradient polarity, for propagating mechanical waves through the subject tissue 152.

In a non-limiting example, the application of a polarizing magnetic field to the subject tissue 152 is performed by the magnet system 110 of MRI system 1000. Additionally or optionally, the irradiation of the subject tissue 152 with the RF excitation field for producing transverse magnetization in spins in the subject tissue 152 is performed by the RF system 130 of MRI system 1000. Additionally or optionally, the application of a magnetic field gradient to the spins which alternates in polarity is performed by the plurality of gradient coils 120 of MRI system 1000. Additionally or optionally, the application of a stress to the subject tissue 152 in cooperation with alternation of the magnetic field gradient polarity for propagating mechanical waves through the subject tissue 152 is performed by the driver 140 of MRI system 1000.

In an exemplary embodiment of step 220, the plurality of operations includes sub-step or step 230. Step 230 includes encoding a series of motions and obtaining MRE data samples in a plurality of directions at a predetermined number of time points. The step 230 of encoding motion and obtaining samples is performed according to an Elastography with Distributed, Generalized Encoding (EDGE) matrix.

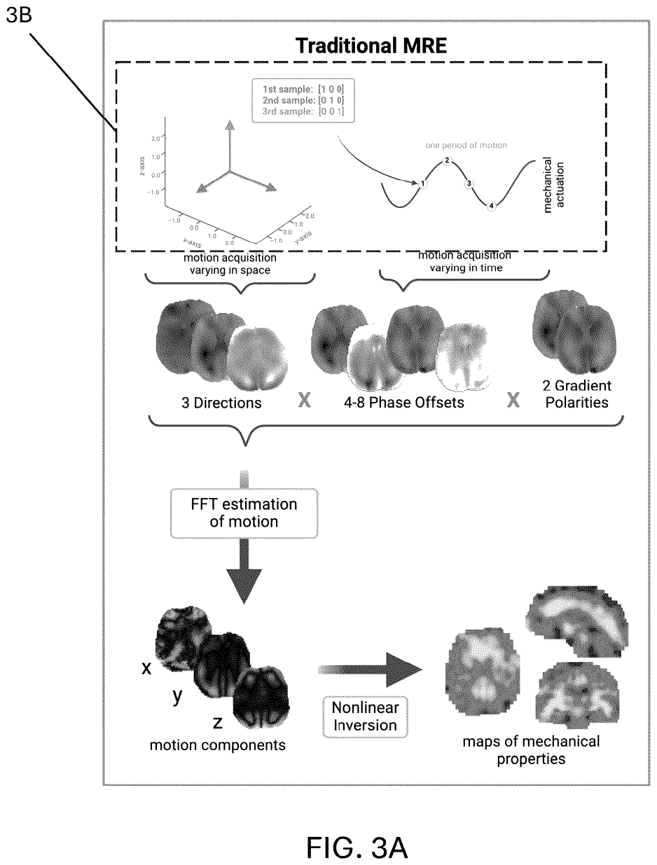

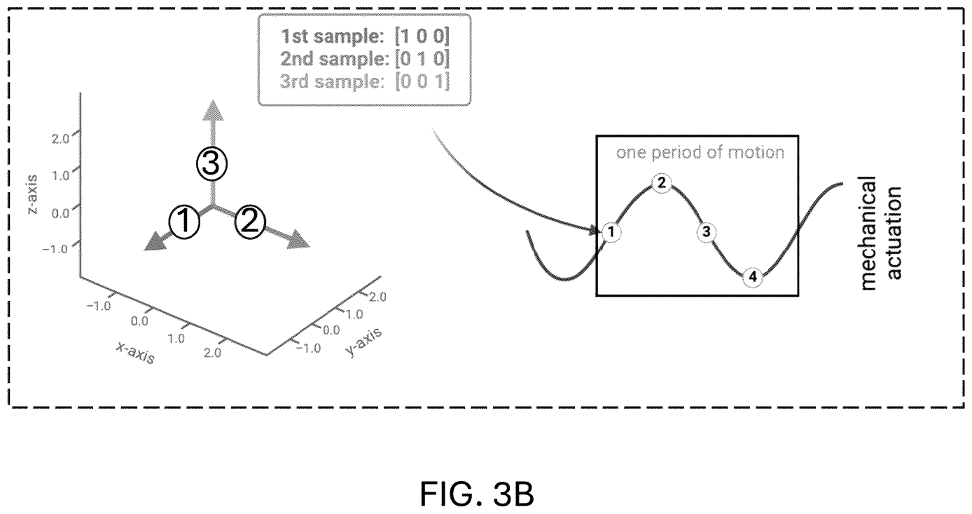

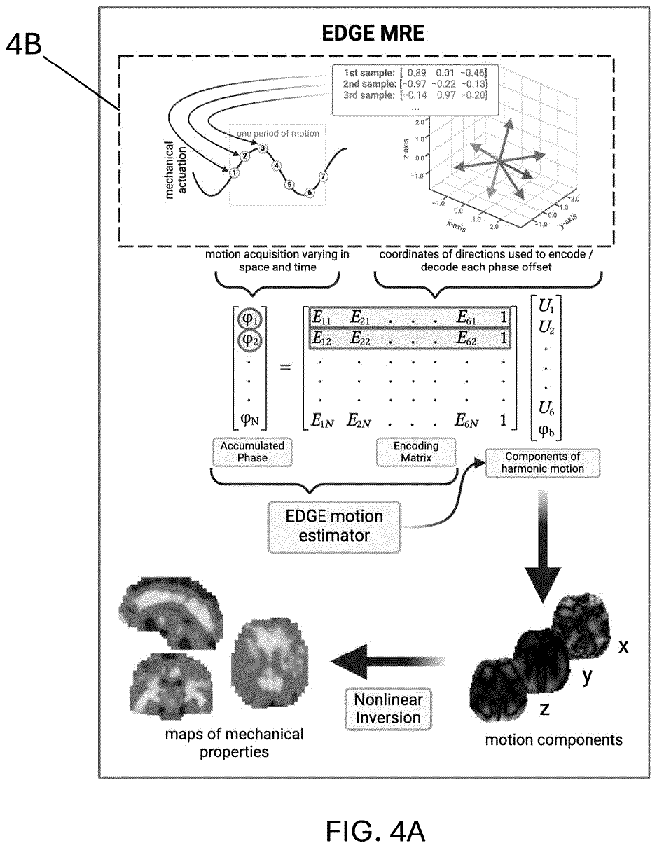

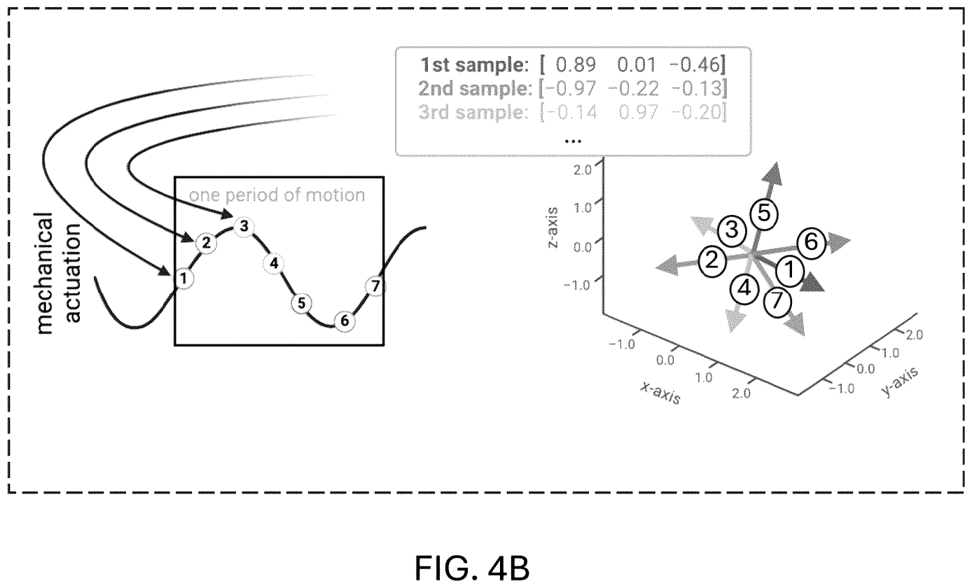

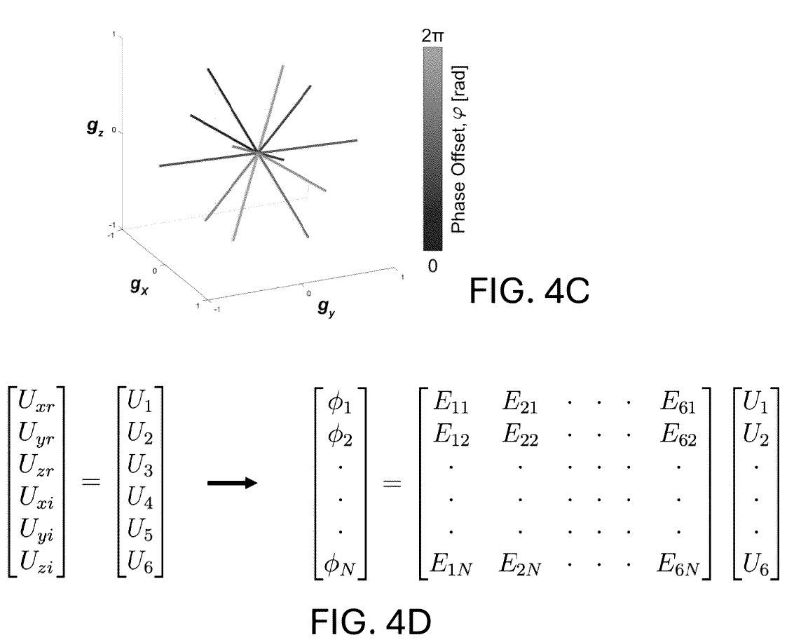

FIGS. 3A-3B depict a generalized encoding matrix in accordance with prior art. In contrast, FIGS. 4A-4D depict an exemplary embodiment of a generalized encoding matrix in accordance with aspects of the invention, and a comparison of FIG. 3A and FIG. 4A, in particular, highlights the contrast between the traditional method in accordance with prior art (FIGS. 3A-3B) and the inventive aspects of method 2000 (FIGS. 4A-4D). Thus, an exemplary embodiment of step 230 includes encoding a series of motions and obtaining MRE data samples along an optimized set of gradient vector directions distributed in a 3-dimensional gradient vector space across a surface of a sphere, as shown, for example in the graph illustrated in FIGS. 4B-4C. The graph in FIGS. 4B-4C shows the encoding motions along the optimized set of gradient vector directions, each being represented by a line on the graph. As illustrated in FIGS. 4A and 4D, each encoding motion (row of the matrix) contains information about each direction (column of the matrix) including both real and imaginary components. Thus, in an exemplary embodiment, the EDGE matrix comprises a generalized encoding matrix that includes encoding motion and obtaining samples along at least one gradient direction (gₓₙ, g_{yn}, g_{zn}) and phase offset (φₙ), as expressed by the following equation:

$${E}_{n} = {MENC}_{n} [ \begin{matrix}{g}_{xn} cos ( {φ}_{n} ) & {g}_{yn} cos ( {φ}_{n} ) & {g}_{zn} cos ( {φ}_{n} ) & - {g}_{xn} sin ( {φ}_{n} ) & - {g}_{yn} sin ( {φ}_{n} ) & - {g}_{zn} sin ( {φ}_{n} ) ]\end{matrix}$$

Additionally, the plurality of operations includes sub-step or step 240. Step 240 includes constructing a plurality of phase images based upon the obtained MRE data samples. In an exemplary embodiment, a series of N phase images from the six complex harmonic bases U (as shown in FIGS. 4A, 4D) is generated based upon the obtained MRE data samples, including a static background phase image (Ø_{b}). In one non-limiting example, the plurality of phase images includes a number of phase images in a range from 7 to 24. In an exemplary embodiment, the number of phase images comprises no more than 7 phase images.

Additionally, the plurality of operations includes sub-step or step 250. Step 250 includes estimating shear displacement fields in the subject tissue 152 based on the plurality of phase images. In an exemplary embodiment, the estimation of shear displacement fields in the subject tissue 152 according to step 250 includes performing an optimization performed in accordance with the EDGE matrix. In this way, the shear displacement fields estimated based on the plurality of phase images constructed based upon the MRE data samples obtained in accordance with the EDGE matrix, ultimately allow for calculation or determination of mechanical properties of the subject tissue 152 in a non-invasive manner. Additionally or optionally, the optimization comprises a predetermined regularization penalty.

Additionally, the plurality of operations includes sub-step or step 260. Step 260 includes generating the map indicative of the subject tissue's 152 mechanical properties via an inversion algorithm. In an exemplary embodiment, the inversion algorithm comprises a non-linear inversion algorithm. Additionally or optionally, the map indicative of the subject tissue's 152 mechanical properties comprises a stiffness map. One skilled in the art would understand from the description herein that the measured, observed, or calculated mechanical properties reflect or are indicative of the microstructural conditions, composition, and organization of the subject tissue, which can have diagnostic implications for assessing cognitive function and overall neural health.

In an exemplary embodiment, sub-step or step 260 comprises generating the stiffness map. Generating the stiffness map includes using at least 7 phase images. In a non-limiting example, the at least 7 phase images are fewer than the number of phase images constructed from sub-step or step 240. In another non-limiting example, the number of phase images is half or less than half a respective number of phase images required for using a traditional MRE technique comprising sampling displacement along three orthogonal directions with four to eight phase offsets and two gradient polarities. In this way, an elapsed time for obtaining the MRE data samples is half or less than half a respective elapsed time required for using a traditional MRE technique comprising sampling displacement along three orthogonal directions with four to eight phase offsets and two gradient polarities. Thus, sub-step or step 260 comprises generating the map having a normalized-root-mean-square error (NRMSE) of 7.5E-9 or less with simulated data or an average difference of 2% across all levels of sampling with real data, as compared to a respective map generated using the traditional MRE technique.

Additionally or optionally, method 2000 includes discarding one or more of the constructed phase images due to artifacts in the one or more discarded phase images. In a non-limiting example, the artifacts are due to subject motion.

Additionally or optionally, sub-step or step 230 is performed such that the EDGE matrix dictates a predetermined sequence and a projected number of phase images, and wherein sub-step or step 260 includes generating the map using at least 7 phase images. The at least 7 phase images are fewer than the projected number of phase images and define a truncation of the predetermined sequence dictated by the EDGE matrix. In a non-limiting example, the truncation is due to subject motion or subject intolerance. Thus, the methods and systems described herein, such as method 2000 and system 1000 described herein, provides a novel and inventive sampling technique and estimation scheme that distributes encodings of direction and time across samples to reduce the number of samples needed to estimate complex, full vector displacement fields.

### Example

The co-inventors assessed the exemplary systems and methods as disclosed herein in a clinical or laboratory setting, to validate feasibility and functionality of the components of the subject systems, as well as verified any updates or improvements made.

In general, the inventive methods and systems described herein is different from conventionally acquired data, which uses Fourier transform method (requires individually sampled directions and time points) and thus requires a “full” sampling due to Nyquist sampling requirements inherent to conventional techniques. However, improvements are desired to accelerate acquisition of data and/or retrospectively reject samples distorted by motion (e.g. of subject during imaging), for example. Thus, as shown in FIGS. 4A-4D, for example, the direction of each sample is described by a line on the graph, and each sample is a row in the matrix (E). Each encoding (row of the matrix) contains information about each direction (column of the matrix) including both real and imaginary components.

Generally, the assessments performed by the co-inventors involved encoding samples along an optimized set of directions distributed uniformly across the surface of a sphere and each sample included information about displacement from all three axes, thereby increasing the information acquired per sample. In this way, fewer samples are required to generate an image (e.g. undersampling) because each sample includes more robust information about the motion at each point. Full set of samples included data with sufficient sampling at both high and low sensitivity to overcome issues with excessive motion. Finally, estimation of motion was performed as an optimization problem minimizing the objective function.

### Materials and Methods

Conventional MRE separately samples displacement along three orthogonal directions with four to eight phase offsets to capture wave propagation through tissue with time, resulting in a total of 24 to 48 images. See e.g., Manduca, A. et al. “Magnetic resonance elastography: Non-invasive mapping of tissue elasticity.” Med. Image Anal. 5, 237-254 (2001) and Nir, G., et al. “A framework for optimization-based design of motion encoding in magnetic resonance elastography.” Magn. Reson. Med. 73, 1514-1525 (2015). Thus, the total sampling is in excess of the theoretical minimum (e.g., seven) in order to account for real and imaginary components of three directions of motion, plus background phase because of how each direction is sampled separately in time and processed via Fourier transforms to extract harmonic motion fields.

However, the inventive encoding matrix, as generated and shown in FIGS. 4A-4D, does not adhere to the traditional sampling pattern, but rather encodes along an optimized set of directions that are distributed uniformly across the surface of a sphere, as can be typical in diffusion tensor imaging. See e.g., Jones, D. K., et al. “Optimal strategies for measuring diffusion in anisotropic systems by magnetic resonance imaging.” Magn. Reson. Med. 42, 515-525 (1999). In this way, every sample includes information from all three cardinal axes, thus contributing information relevant to estimating each displacement of the subject tissue in response to mechanical actuation and in each direction. Eschewing the traditional Fourier technique, the estimation of motion is formulated from the obtained samples as an optimization problem using a general encoding matrix and minimizing the objective function, which follows analogous examples in field mapping by Fessler. See e.g., Funai, A. K., Fessler, J. A., Yeo, D. T. B., Noll, D. C. & Olafsson, V. T. “Regularized field map estimation in MRI.” IEEE Trans. Med. Imaging 27, 1484-1494 (2008) and Lin, C. Y. & Fessler, J. A. “Efficient Regularized Field Map Estimation in 3D MRI.” IEEE Trans. Comput. Imaging 6, 1451-1458 (2020).

The feasibility and performance of the inventive encoding matrix (e.g. the Elastography with Distributed, Generalized Encoding (EDGE) matrix) was assessed through retrospective sampling of realistic simulated brain data using the inventive EDGE sampling scheme. A set of 24 phase images was used, which mimics the total number of images typically acquired in a traditional MRE, and then reducing down to the theoretical minimum of seven phase images. See e.g., Lin, C. Y. as cited above and Trzasko, J. et al. “Theoretical Performance and Sampling Limits in Steady-State Magnetic Resonance Elastography.” in International Society of Magnetic Resonance in Medicine 2518 (2015). Stiffness maps were generated using a nonlinear inversion algorithm. See e.g., McGarry, M. D. J. et al. “Multiresolution MR elastography using nonlinear inversion.” Med. Phys. 39, 6388-6396 (2012). Stiffness maps were compared with traditionally sampled and processed MRE data using normalized-root-mean-square error (NRMSE).

### Results of Simulated Data

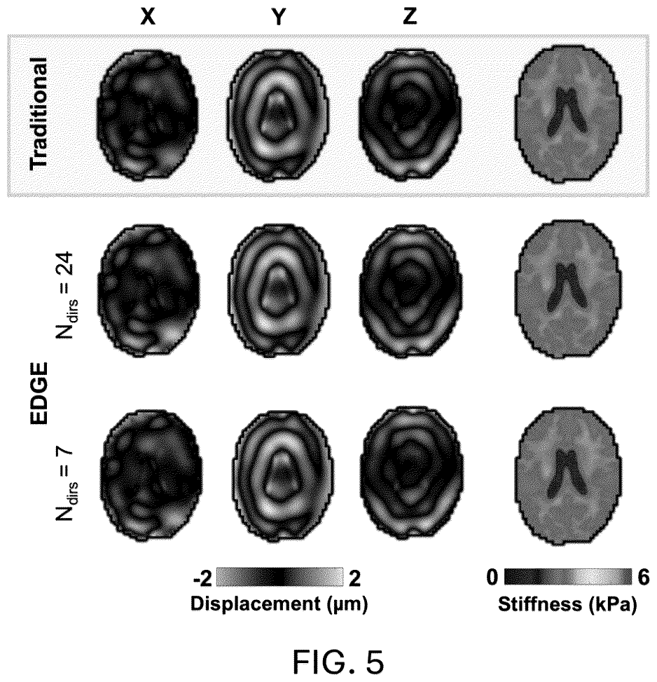

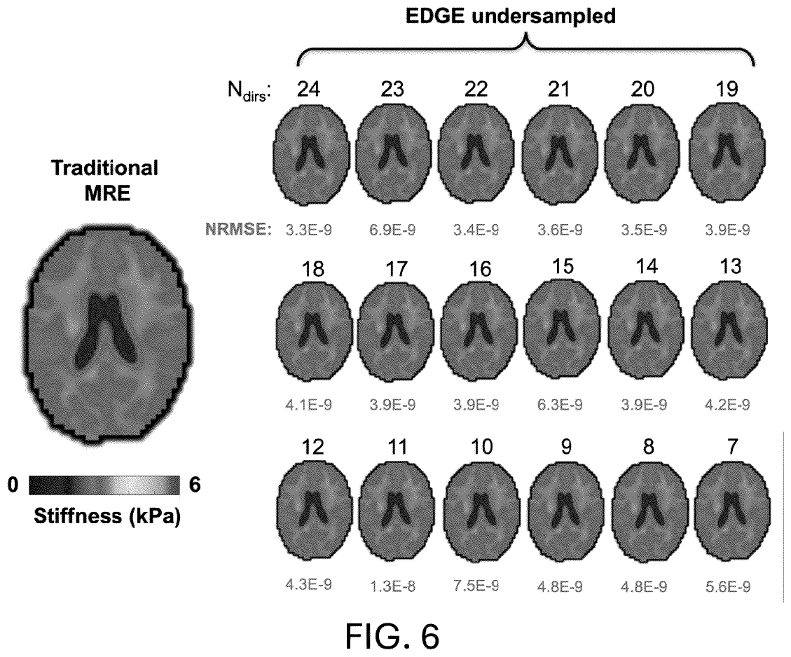

Using simulated data, it was discovered that the inventive methods and systems described herein can accurately recover original displacement fields and stiffness maps with high quality and agreement with traditional MRE even after high levels of undersampling, as indicated, for example, by FIG. 5. Briefly, the displacement maps in all three orthogonal directions generated with the inventive methods and systems described herein of simulated brain data show near-identical wave motion to standard sampling. Further, as shown in FIG. 6, reduction of sampling using the inventive methods and systems described herein provides good or suitable agreement with traditional methods for generation of stiffness maps across a range of undersampling levels. FIG. 6, for example, shows simulated brain data with all available degrees of undersampling (N_{dirs}=24 to 7) with the NRMSE calculated for the whole image (30 slices at 2.5 mm³ resolution). The results from FIG. 6 indicate that undersampling can be performed at any interval, to a minimum of seven with a calculated NMRSE of 5.6×10⁻⁹, with an average NRMSE of 4.9×10⁻⁹ across all levels of undersampling.

Notably, generating the map indicative of the subject tissue's mechanical properties via an inversion algorithm can be performed relatively efficiently, taking less than 3 minutes to reconstruct a full 3D dataset on a desktop computer, and using theoretical minimum sampling which provides 3.4 times shorter scan acquisition time. Further, the inventive encoding matrix may be implemented in any current MRE sequence to collect data with reduced sampling in human subjects, thereby mitigating or preventing issues arising from motion of the subject during imaging for MRE acquisition and data quality. Successful implementation of the inventive methods and systems described herein will enable prospective acceleration or retrospective volume rejection—or a combination of both—for uncooperative subject or participants, significantly advancing the adoptability of MRE.

### Results of Real Phantom Data and Real Human Data

An Echo-planar imaging (EPI) MRE sequence incorporating the inventive methods and systems described herein using optimized direction sets was implemented. Undersampling allowed for acceleration up to 58 seconds for 40 slices of 2.5 mm isotropic data (from a non-limiting example of original scan time of 2 mins 19 s). Testing in both phantoms and humans showed that the inventive methods and systems described herein perform comparably to conventional MRE methods and systems. It is important to note that with real data there will be several orders of magnitude greater difference between data acquired with the inventive methods and the reference traditional acquisition methods as compared to that seen with simulated data. This is because the simulated data is retrospectively sampled with the inventive methods meaning that the motion being sampled is identical every time. The real data is sampled prospectively with the inventive directions, meaning that there is variation in the motion between each sample. In general, with brain MRE techniques, 2-3% difference can be expected with repeated measurements, so that much difference between measurements is expected, plus any additional differences from the implementation of the inventive technique.

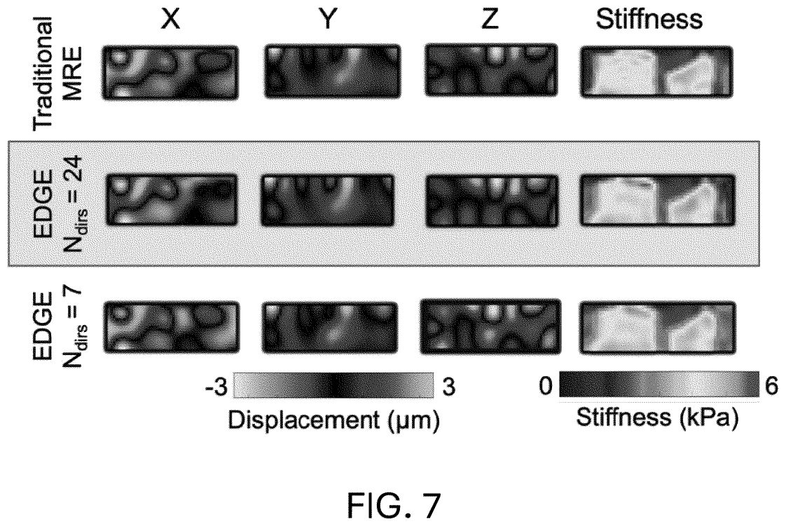

As shown in FIG. 7, an agarose phantom with stiff inclusions was created and scanned with the inventive methods and systems described herein and showed reliable reconstruction of stiffness maps with good spatial resolution and low error with sampling reduced to minimum. For example, phantom stiffness maps created and scanned with the inventive methods and systems described herein (with a number of phase images at N_{dirs}=24) had NRMSE of 6.5% compared to the conventional MRE data (with a number of phase images at N_{dirs}=24).

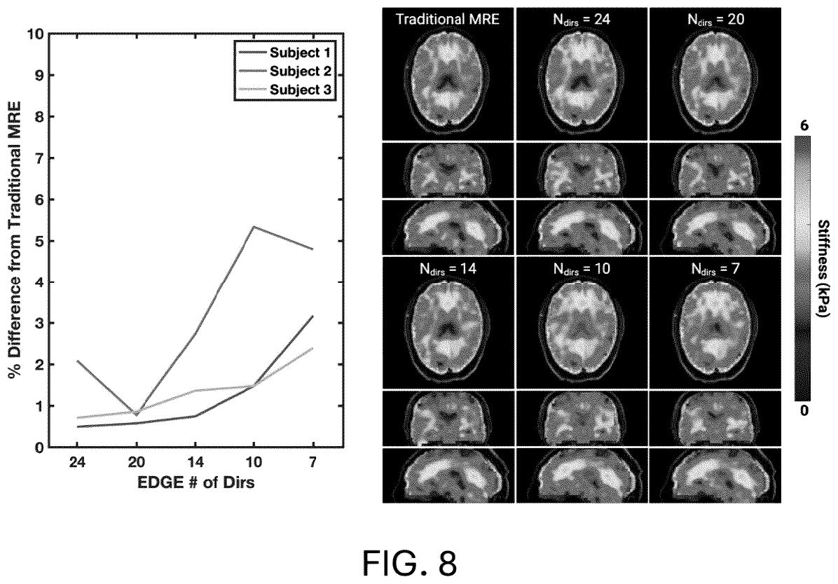

As shown in FIG. 8, three human subjects, each in a single scanning session, were scanned with conventional MRE methods and with the inventive methods and systems described herein. Data reconstructed with both techniques show good or suitable agreement proving reliability of the technique for human brain applications. For example, FIG. 8 shows data obtained at acquisition time (TA) or duration of less than one minute (e.g. 58 seconds) with a number of phase images at N_{dirs}=7, showing only 3.2% difference from data obtained via conventional MRE methods, with an average 2% difference from traditional methods across all subjects and all levels of sampling. This difference is largely attributed to the normal variation that occurs when repeating scans in a single scanning session. Another contributing factor to the disagreement of the two stiffness maps is due to the roughness penalty used for a regularization term, which may be optimized to stabilize the results and minimize the effects of noise, thereby further improving agreement between stiffness maps generated with the inventive methods and systems described herein in comparison with conventionally collected MRE data. Additional parameter optimization can be accomplished through optimization of the inventive encoding matrix so that sampling of unique directions and time points can be done with maximum efficiency.

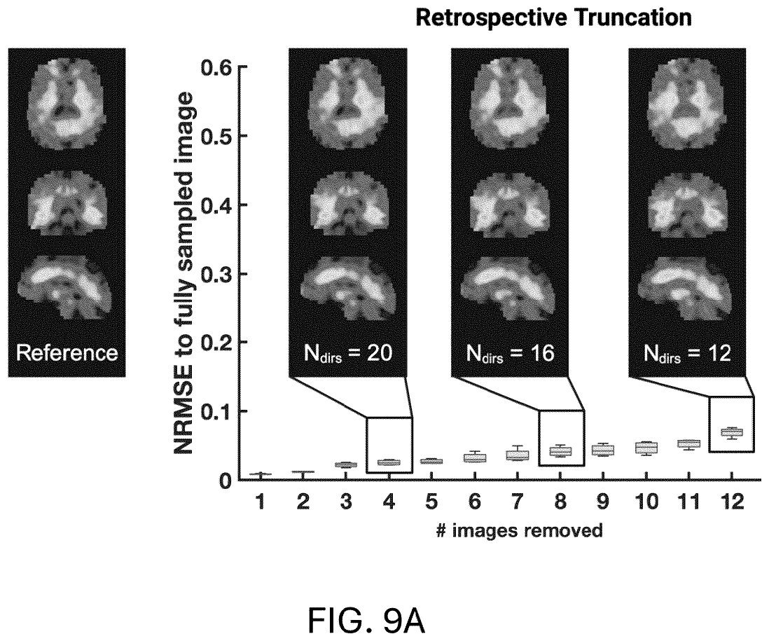

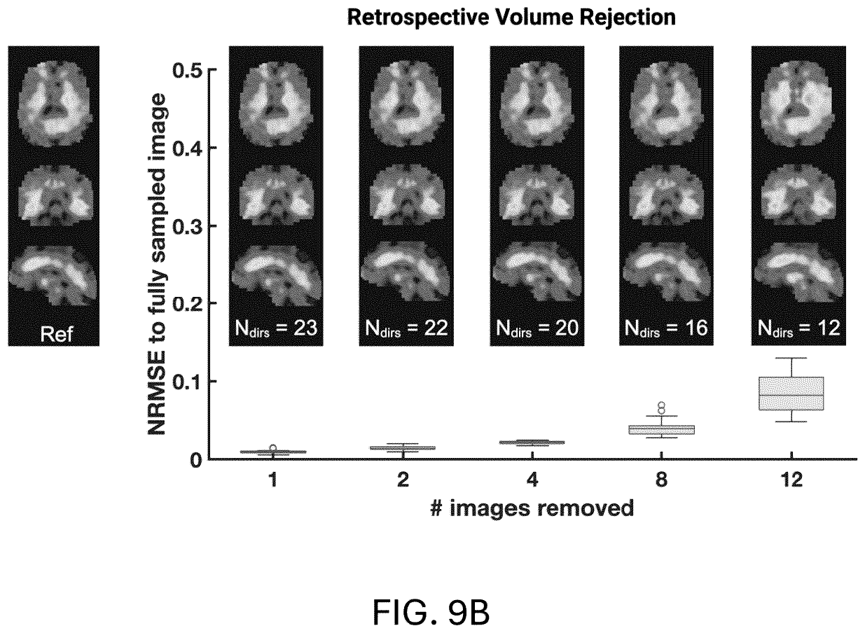

Thus, the inventive methods and systems described herein allow for MRE scans to be collected to create high quality stiffness maps efficiently, such as under a minute (40 slices, 2.5 mm³ resolution), for example. Further, the inventive methods and systems described herein permit undersampling (e.g. a lower minimum of samples needed to generate an image) prospectively to shorten scan time, or retrospectively to remove artifacts caused by subject motion during imaging. Further optimization of encoding directions can be performed, including improvements related to volume rejection capabilities, truncation for subject noncompliance, and mid-scan volume rejection for subject motion. Volume rejection and truncation capabilities are demonstrated in FIGS. 9A-9B. In this case, both volume rejection and truncation were performed retrospectively to fully sampled EDGE data (N_{dirs}=24) and samples were removed from the input either from the end of the scan chronologically in the case of truncation, or randomly in the case of volume rejection. For truncation (FIG. 9A), samples were removed from 24 sampled images down to 12 images remaining images. Each amount of rejection was tested on four available fully sampled scans from the same subject. These were tested with volume rejection (FIG. 9B) in intervals, leaving 23, 22, 20, 16, and 12 images for the motion to be estimated from and then the resulting stiffness map was generated using normal methods. The volume rejection testing was done 24 times for each level of rejection. The NRMSE for all retrospective reduction was less than 0.2, which shows that the inventive methods and systems described herein can be used even in cases that previously would render traditional MRE scans useless (e.g., due to sporadic subject motion or subject intolerance leading the scan to be cut short, etc.). Further optimization includes testing different inversion methods to avoid issues at boundaries and optimization of regularization parameters.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A method for generating a map indicative of mechanical properties of a subject tissue using a magnetic resonance imaging (MRI) system, the method comprising:
performing a magnetic resonance elastography (MRE) scan on the subject tissue with the MRI system;
obtaining and processing MRE data related to the subject tissue via a control device coupled to the MRI system, the control device comprising a controller configured to perform a plurality of operations in accordance with machine readable instructions stored in a digital memory, wherein the plurality of operations includes:
encoding a series of motions and obtaining MRE data samples in a plurality of directions at a predetermined number of time points, the encoding motion and obtaining samples performed according to an Elastography with Distributed, Generalized Encoding (EDGE) matrix,
constructing a plurality of phase images based upon the obtained MRE data samples, the plurality of phase images comprising a number of phase images in a range from 7 to 24,
estimating shear displacement fields in the subject tissue based on the plurality of phase images, wherein estimation comprises an optimization performed according to the EDGE matrix, and
generating the map indicative of the subject tissue's mechanical properties via an inversion algorithm.

2. The method of claim 1, wherein encoding a series of motions comprises encoding and obtaining samples along an optimized set of gradient vector directions distributed in a 3-dimensional gradient vector space across a surface of a sphere.

3. The method of claim 1, wherein optimization comprises a predetermined regularization penalty.

4. The method of claim 1, wherein the inversion algorithm comprises a non-linear inversion algorithm.

5. The method of claim 1, wherein the number of phase images comprises no more than 7 phase images.

6. The method of claim 1, wherein the map indicative of the subject tissue's mechanical properties comprises a stiffness map.

7. The method of claim 1, wherein performing the MRE scan comprises:
applying a polarizing magnetic field to the subject tissue;
irradiating the subject tissue with a radio frequency (RF) excitation field for producing transverse magnetization in spins in the subject tissue;
applying a magnetic field gradient to the spins which alternates in polarity; and
applying a stress to the subject tissue in cooperation with alternation of the magnetic field gradient polarity, for propagating mechanical waves through the subject tissue.

8. The method of claim 1, wherein the EDGE matrix comprises a generalized encoding matrix comprises encoding motion and obtaining samples along at least one gradient direction (gₓₙ, g_{yn}, g_{zn}) and phase offset (φₙ), as expressed by the following equation:
$${E}_{n} = {MENC}_{n} [ \begin{matrix}{g}_{xn} cos ( {φ}_{n} ) & {g}_{yn} cos ( {φ}_{n} ) & {g}_{zn} cos ( {φ}_{n} ) & - {g}_{xn} sin ( {φ}_{n} ) & - {g}_{yn} sin ( {φ}_{n} ) & - {g}_{zn} sin ( {φ}_{n} ) ]\end{matrix}$$

9. A magnetic resonance imaging (MRI) system for generating a map indicative of a subject tissue's mechanical properties, the system comprising:
a magnet system configured to generate a polarizing magnetic field to the subject tissue;
a plurality of gradient coils configured to apply a magnetic field gradient to the polarizing magnetic field;
a radio frequency (RF) system configured to apply an excitation field to the subject tissue;
a driver configured to deliver a stress to the subject for propagating mechanical waves through the subject tissue;
a control device coupled to the MRI system, the control device comprising a controller configured to perform a plurality of operations in accordance with machine readable instructions stored in a digital memory, wherein the plurality of operations includes:
encoding a series of motions and obtaining MRE data samples in a plurality of directions at a predetermined number of time points, the encoding motion and obtaining samples performed according to an Elastography with Distributed, Generalized Encoding (EDGE) matrix, comprising encoding and obtaining samples along an optimized set of gradient vector directions distributed in a 3-dimensional gradient vector space across a surface of a sphere,
constructing a plurality of phase images based upon the obtained MRE data samples, the plurality of phase images comprising a number of phase images in a range from 7 to 24,
estimating shear displacement fields in the subject tissue based on the plurality of phase images, wherein estimation comprises an optimization performed according to an Elastography with Distributed, Generalized Encoding (EDGE) matrix, and
generating the map indicative of the subject tissue's mechanical properties via an inversion algorithm.

10. The method of claim 1, wherein generating the map comprises generating a stiffness map.

11. The method of claim 10, comprising generating the map using at least 7 phase images, wherein the at least 7 phase images are fewer than the number of phase images constructed.

12. The method of claim 11, comprising discarding one or more of the constructed phase images due to artifacts in the one or more discarded phase images.

13. The method of claim 12, wherein the artifacts are due to subject motion.

14. The method of claim 10, wherein the EDGE matrix dictates a predetermined sequence and a projected number of phase images, wherein generating the map comprises using at least 7 phase images, wherein the at least 7 phase images are fewer than the projected number of phase images, and define a truncation of the predetermined sequence.

15. The method of claim 14, wherein the truncation is due to subject motion or subject intolerance.

16. The method of claim 10, wherein the number of phase images is half or less than half a respective number of phase images required for using a traditional MRE technique comprising sampling displacement along three orthogonal directions with four or eight phase offsets and two gradient polarities.

17. The method of claim 16, wherein an elapsed time for obtaining the MRE data samples is half or less than half a respective elapsed time required for using a traditional MRE technique comprising sampling displacement along three orthogonal directions with four to eight phase offsets and two gradient polarities.

18. The method of claim 17, comprising generating the map having a normalized-root-mean-square error (NRMSE) of 7.5E-9 or less with simulated data.

19. The method of claim 17, comprising generating the map having an average difference of 2% across all levels of sampling with real data, as compared to a respective map generated using the traditional MRE technique.
